SEQUENCE LISTING (4 sequences)

SEQ ID NO: 1 - DNA, 798 bp, Artificial Sequence
  Synthetic construct
  misc_feature (139)..(140): n is a, c, g, or t
  misc_feature (145)..(146): n is a, c, g, or t
  misc_feature (148)..(149): n is a, c, g, or t
  misc_feature (205)..(206): n is a, c, g, or t
  misc_feature (211)..(212): n is a, c, g, or t
  misc_feature (214)..(215): n is a, c, g, or t
gaaaccattaccgtgagcaccccgatcaaacagatttttccggatgacgcgttcgccgaaacgatcaaagcaaacctgaagaaaaagagcgttaccgatgctgtcacgcaaaatgaactgaacagtattgaccagatcnnkgcgnnknnktccgatatcaaatcagtgcaaggcattcagtatctgccgaatgttcgttacctgnnkctgnnknnkaacaaactgcatgacatctcggcactgaaagaactgaccaatctgacgtatctgattctgaccggtaaccaactgcagagcctgccgaatggcgtctttgataaactgacgaacctgaaagaactggtgctggttgaaaatcaactgcagtctctgccggacggtgtcttcgataaactgaccaacctgacgtacctgaatctggctcacaaccaactgcagagtctgccgaaaggcgtgtttgacaaactgaccaatctgacggaactggatctgtcctataaccaactgcagtcactgccggaaggtgttttcgacaaactgacccagctgaaagatctgcgcctgtaccagaatcagctgaaatcggtcccggacggcgtgtttgatcgtctgaccagcctgcagtatatctggctgcatgataacccgtgggattgcacctgtccgggtattcgctacctgtctgaatggatcaataaacacagtggcgttgtccgtaactccgcgggttcagttgccccggattcggcgaaatgctccggcagcggtaaaccggtgcgtagcattatttgcccgacc

SEQ ID NO: 2 - protein, 266 aa, Artificial Sequence
  Synthetic construct
ETITVSTPIKQIFPDDAFAETIKANLKKKSVTDAVTQNELNSIDQIIANNSDIKSVQGIQYLPNVRYLALGGNKLHDISALKELTNLTYLILTGNQLQSLPNGVFDKLTNLKELVLVENQLQSLPDGVFDKLTNLTYLNLAHNQLQSLPKGVFDKLTNLTELDLSYNQLQSLPEGVFDKLTQLKDLRLYQNQLKSVPDGVFDRLTSLQYIWLHDNPWDCTCPGIRYLSEWINKHSGVVRNSAGSVAPDSAKCSGSGKPVRSIICPT

SEQ ID NO: 3 - protein, 266 aa, Artificial Sequence
  Synthetic construct
ETITVSTPIKQIFPDDAFAETIKANLKKKSVTDAVTQNELNSIDQIIANNSDIKSVQGIQYLPNVRYLALGGNKLHDISALKELTNLTYLNLWENQLQSLPNGVFDKLTNLKELTLFLNQLQSLPDGVFDKLTNLTYLNLAHNQLQSLPKGVFDKLTNLTELDLSYNQLQSLPEGVFDKLTQLKDLRLYQNQLKSVPDGVFDRLTSLQYIWLHDNPWDCTCPGIRYLSEWINKHSGVVRNSAGSVAPDSAKCSGSGKPVRSIICPT

SEQ ID NO: 4 - protein, 266 aa, Artificial Sequence
  Synthetic construct
ETITVSTPIKQIFPDDAFAETIKANLKKKSVTDAVTQNELNSIDQIIANNSDIKSVQGIQYLPNVRYLALGGNKLHDISALKELTNLTYLNLWENQLQSLPNGVFDKLTNLKELTLFLNQLQSLPDGVFDKLTNLTYLNLKFNQLQSLPKGVFDKLTNLTELDLSYNQLQSLPEGVFDKLTQLKDLRLYQNQLKSVPDGVFDRLTSLQYIWLHDNPWDCTCPGIRYLSEWINKHSGVVRNSAGSVAPDSAKCSGSGKPVRSIICPT

(19) United States
(12) Patent Application Publication
(10) Pub. No.: US 2022/0315662 A1
(43) Pub. Date: Oct. 6, 2022

(54) NOVEL POLYPEPTIDE SELECTIVELY BINDING TO HUMAN PROGRAMMED CELL DEATH PROTEIN (PD-1) AND USE THEREOF

(71) Applicant: KOREA ADVANCED INSTITUTE OF SCIENCE AND TECHNOLOGY, Daejeon (KR)

(72) Inventors: Hak-Sung KIM, Daejeon (KR); Sumin SON, Deajeon (KR); Hyo-Deok SEO, Daejeon (KR); Jinho PARK, Daejeon (KR)

(21) Appl. No.: 17/637,377

(22) PCT Filed: Aug. 21, 2020
(86) PCT No.: PCT/KR2020/011193

§ 371 Date: Feb. 22, 2022

(30) Foreign Application Priority Data
Aug. 23, 2019 (KR) 10-2019-0103737

(51) Int. Cl. C07K 16/28; A61P 35/00; C12N 15/63

(52) U.S. Cl. CPC C07K 16/2896; A61P 35/00; C12N 15/63; A61K 2039/505

(57) ABSTRACT

The present invention relates to a novel polypeptide which can specifically bind to programmed cell death protein 1, a polynucleotide coding for the polypeptide, a vector comprising the polynucleotide, a recombinant microorganism in which the expression vector has been introduced, a method for preparing the polypeptide by means of the recombinant microorganism, a cancer preventing or treating composition comprising the polypeptide, and a cancer prevention or treatment method comprising administration of the cancer preventing or treating composition comprising the polypeptide. The polypeptide of the present invention can inhibit the activity of programmed cell death protein 1 by binding thereto and thus can be widely utilized for a formulation for preventing or treating various diseases associated with programmed cell death protein 1.

### TECHNICAL FIELD

The present invention relates to a novel polypeptide selectively binding to human programmed cell death protein 1 (PD-1), and more particularly to a polypeptide capable of serving as a cancer immunotherapeutic agent by binding to human programmed cell death protein 1, a polynucleotide encoding the polypeptide, a recombinant vector including the polynucleotide, a recombinant microorganism into which the recombinant vector is introduced, a method of producing the polypeptide using the recombinant microorganism, and a therapeutic pharmaceutical composition containing the polypeptide.

### BACKGROUND ART

First-generation chemotherapy and second-generation targeted therapy, which are currently widely used, are problematic because of side effects due to toxicity of anticancer drugs, high risk of drug tolerance, and administrability only to patients with specific genetic mutations. Third-generation cancer immunotherapy, which overcomes these problems, acts on signaling pathways of immune cells to thus activate immune cells to attack cancer cells, resulting in a therapeutic effect. Unlike existing anticancer drugs, cancer immunotherapy serves to treat cancer cells by activating the immune system of the human body, and is applicable to treatment of various types of cancer, and side effects thereof are also reported to be lower than that of existing anticancer drugs.

Programmed cell death protein 1 is an immune checkpoint receptor, and may be an excellent target protein for cancer immunotherapy in the treatment of various types of cancer, including malignant melanoma, non-small cell lung cancer, renal cell carcinoma, and the like. Programmed cell death protein 1 (PD-1) is a receptor that is present on the surface of T cells and belongs to the immunoglobulin superfamily. When programmed cell death protein 1 binds to a ligand thereof, namely programmed death ligand 1 (PD-L1) or programmed death ligand 2 (PD-L2), signaling involving PD-1 is activated, and proliferation of T cells, production of interferon-gamma and interleukin-2, and signaling of T cell receptors are suppressed, thereby inhibiting activation of T cells. It is known that various types of cancer cells express PD-1, resulting in the loss of function of T cells, which acts as a mechanism for cancer cells to evade immune system attacks.

Drugs that target PD-1 include nivolumab and pembrolizumab as monoclonal antibodies, and these drugs are used as therapeutic agents for malignant melanoma and non-small cell lung cancer, but are reported to impose a high economic burden on patients due to the high production cost thereof and to require accurate verification of therapeutic efficacy thereof. Therefore, there is an urgent need for the development of a novel PD-1-targeting therapeutic agent that overcomes the limitations of existing drugs.

The present inventors have developed a polypeptide called a repebody that is able to specifically bind to various proteins (Korean Patent Nos. 10-1356075, 10-1654890, 10-1587622, 10-1624702, 10-1624703, 10-1713944, 10-1751501, 10-1818152, and 10-1875809).

A repebody is a polypeptide optimized by fusing the N-terminus of internalin B, having an LRR (leucin-rich repeat) structure, with VLR based on the structural similarity therebetween so as to realize a consensus design. The repebody has a size corresponding to ⅕ of the size of a typical antibody, and may be mass-produced in E. coli, so the production cost thereof may be lowered, and industrial application thereof is favorable because of the excellent thermal and pH stability thereof. Moreover, it has been proven that the repebody is capable of easily increasing binding affinity to a target material and that specificity thereof to the target material is vastly superior.

Against this background, the present inventors have made great efforts to successfully obtain a protein that specifically binds to PD-1, which is an important target protein for the treatment of various types of cancer, using the repebody scaffold, produced and selected a novel polypeptide having specific binding affinity to PD-1 based on a random mutation library constructed through analysis of overall structure and modular structural characteristic of the repebody, and ascertained that the repebody selectively binds to PD-1 to thus inhibit interaction with the ligand PD-L1, thereby activating immune cells and inhibiting proliferation of cancer cells, thus culminating in the present invention.

### DISCLOSURE

It is an object of the present invention to provide a repebody specifically binding to human PD-1, a polynucleotide encoding the repebody, a recombinant vector including the polynucleotide, a recombinant microorganism into which the recombinant vector is introduced, and a method of producing the repebody using the recombinant microorganism.

It is another object of the present invention to provide a composition for preventing or treating cancer containing the polypeptide and a method of preventing or treating cancer including administering the composition for preventing or treating cancer containing the polypeptide.

In order to accomplish the above objects, the present invention provides a polypeptide selectively binding to programmed cell death protein 1 (PD-1), in which the N-terminus of an LRR (leucine-rich repeat) family protein having an alpha-helical capping motif, the modified repeat module of a VLR (variable lymphocyte receptor) protein, and the C-terminus of the VLR protein are fused, including one or more amino acid mutations selected from the group consisting of, in the amino acid sequence represented by SEQ ID NO: 2, i) substitution of isoleucine at position 91 with asparagine, ii) substitution of threonine at position 93 with tryptophan, iii) substitution of glycine at position 94 with glutamic acid, iv) substitution of valine at position 115 with threonine, v) substitution of valine at position 117 with phenylalanine, vi) substitution of glutamic acid at position 118 with leucine, vii) substitution of alanine at position 141 with lysine, and viii) substitution of histidine at position 142 with phenylalanine.

In addition, the present invention provides a polynucleotide encoding the polypeptide and a recombinant vector including the polynucleotide.

In addition, the present invention provides a recombinant microorganism including the recombinant vector.

In addition, the present invention provides a method of producing a polypeptide including expressing the polypeptide by culturing the recombinant microorganism and recovering the polypeptide from the cultured recombinant microorganism or a culture.

In addition, the present invention provides a composition for preventing or treating cancer containing the polypeptide as an active ingredient.

In addition, the present invention provides a method of treating or preventing cancer including administering the polypeptide or a pharmaceutically acceptable salt thereof.

In addition, the present invention provides the use of the polypeptide or a pharmaceutically acceptable salt thereof for the treatment or prevention of cancer.

In addition, the present invention provides the use of the polypeptide or a pharmaceutically acceptable salt thereof for the manufacture of a medicament for the treatment or prevention of cancer.

### DESCRIPTION OF DRAWINGS

FIG. 1 schematically shows the overall structure of a repebody indicating amino acid residues constructing a random library in order to select a polypeptide that specifically binds to PD-1;

FIG. 2 shows the results of measurement, using isothermal titration calorimetry (ITC), of the binding affinity of a repebody clone primarily selected after biopanning of a phage display for PD-1 using a phage library constructed in the present invention;

FIG. 3 shows the results of measurement, using isothermal titration calorimetry (ITC), of the binding affinity of a repebody clone finally selected after biopanning of a phage display for PD-1 using a phage library constructed to increase binding affinity from the primarily selected repebody clone;

FIG. 4 shows results confirming specific binding of the G9 repebody selected in the present invention to human PD-1;

FIG. 5 shows results of inhibition of binding of PD-1 and PD-L1 due to action of the G9 repebody selected in the present invention at different concentrations as a competitor to PD-L1;

FIG. 6 shows experimental results confirming production of interleukin-2 (IL-2), which is a cytokine used as an indicator of T-cell activation, in a mixed lymphocyte reaction (MLR) test by treating T cells isolated from human peripheral blood mononuclear cells and allogeneic dendritic cells with the G9 repebody selected in the present invention at different concentrations;

FIG. 7 shows experimental results confirming production of interferon-gamma (interferon-γ (IFN-γ)), which is a cytokine used as an indicator of T-cell activation, in a mixed lymphocyte reaction (MLR) test by treating T cells isolated from human peripheral blood mononuclear cells and allogeneic dendritic cells with the G9 repebody selected in the present invention at different concentrations;

FIG. 8 shows experimental results confirming inhibition of cancer cell proliferation when the G9 repebody selected in the present invention is injected into an immune-deficient mouse model inoculated with human peripheral blood mononuclear cells and NCI-H292 (lung cancer cell line) cells; and

FIG. 9 shows experimental results confirming significant reduction of the tumor size on the 31^{st} day in a mouse model experiment using the G9 repebody selected in the present invention, DPBS indicating a control group, r_off indicating an off-target wild-type repebody control group, and r_G9 indicating an experimental group treated with the repebody of the present invention.

### MODE FOR INVENTION

Unless otherwise defined, all technical and scientific terms used herein have the same meanings as those typically understood by those skilled in the art to which the present invention belongs. In general, the nomenclature used herein is well known in the art and is typical.

In the present invention, the present inventors intended to select a polypeptide that specifically binds to programmed cell death protein 1 (PD-1) and to confirm the effect of activating T cells by inhibiting the activity of PD-1.

In the present invention, operations of selecting a polypeptide having binding specificity, among polypeptides included in a library for repebody development, through biopanning using a phagemid of PD-1 and of improving the binding affinity thereof were performed. Thereby, a polypeptide specific to PD-1 was produced.

In an embodiment of the present invention, in order to develop a novel polypeptide capable of specifically binding to PD-1, a library randomly including the repeat module of the polypeptide, in which the N-terminus of an internalin B protein and the LRR (leucine-rich repeat) protein portion of a variable lymphocyte receptor (VLR) were fused, was constructed (FIG. 1). The polypeptide included in the library may be encoded by the polynucleotide sequence of SEQ ID NO: 1, or may be encoded by a polynucleotide sequence having homology of 75%, preferably 85%, more preferably 90%, and much more preferably 95% or more with the polynucleotide sequence of SEQ ID NO: 1.

Also, the library may take the form of a phagemid including the polynucleotide. As used herein, the term “phagemid” refers to a circular polynucleotide molecule derived from a phage, which is a virus having E. coli as a host, and includes sequences of proteins and surface proteins necessary for propagation and proliferation. A recombinant phagemid may be produced using gene recombinant technology well-known in the art, and site-specific DNA cleavage and ligation may be performed using enzymes or the like generally known in the art. The phagemid may include a signal sequence or leader sequence for secretion, in addition to expression regulatory factors such as a promoter, operator, initiation codon, termination codon, and enhancer, and may be mainly used in a method of labeling the protein on a phage surface by fusing a desired protein with a surface protein of the phage. The promoter of the phagemid is primarily inducible, and may include a selective marker for selecting a host cell. For the purpose of the present invention, the phagemid may be the polynucleotide of SEQ ID NO: 2, described in a patent previously granted to the present inventors (No. 10-2012-0019927), which includes MalEss, DsbAss, or PelBss, as a signal sequence or leader sequence for expressing and secreting a polynucleotide encoding a polypeptide constituting the library, and includes a histidine-tag for confirming expression of a recombinant protein on the surface of the phage and a polynucleotide encoding a gp3 domain, which is a type of surface protein of M13 phage for expression on the surface of the phage, but the present invention is not particularly limited thereto.

The term “programmed cell death protein 1” as used in the present invention is interchangeable with CD279 (cluster of differentiation 279), refers to a signaling protein called an immune checkpoint, and is known to play a role of regulating the activation and function of T cells. This protein is a membrane protein consisting of 268 amino acids, and is composed of an extracellular IgV domain, a transmembrane domain, and an intracellular tail. The binding of PD-1 of T cells to PD-L1, which is a ligand expressed from cancer cells, in the tumor microenvironment activates the PD-1 signaling pathway, leading to inactivation of T cells. This phenomenon has been observed in various types of cancer, such as malignant melanoma, non-small cell lung cancer, renal cell cancer, and the like.

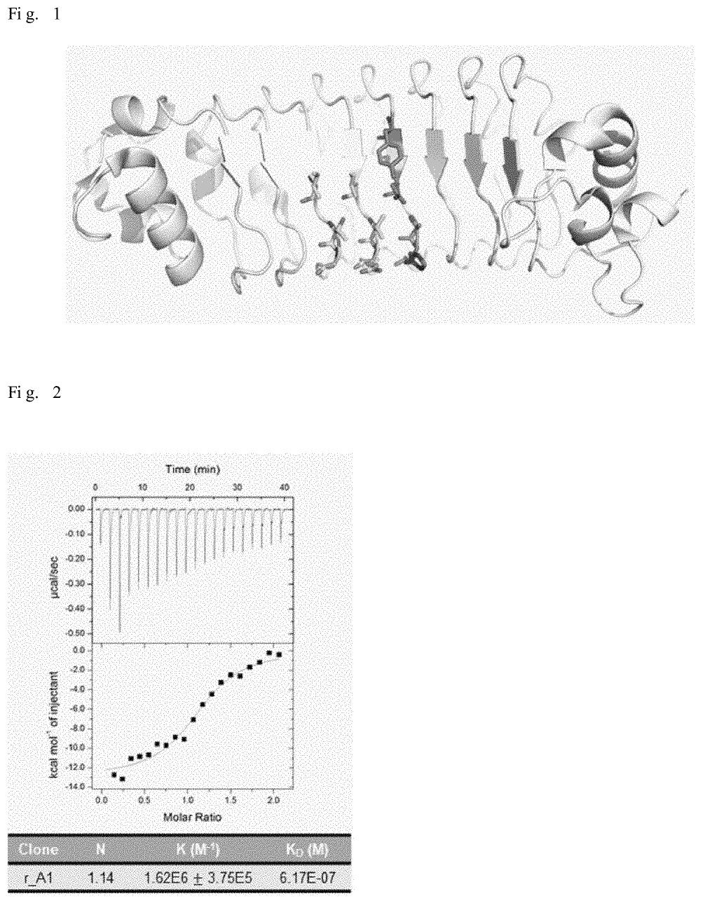

The present inventors selected a novel polypeptide (SEQ ID NO: 3) having the form of a repebody having high ability to bind to PD-1 by mutating amino acids at one or more positions selected from the group consisting of amino acid positions 91, 93, 94, 115, 117, and 118 in the amino acid sequence encoded by the DNA sequence of SEQ ID NO: 1 through a phage display method using the library including the phagemid (FIG. 2).

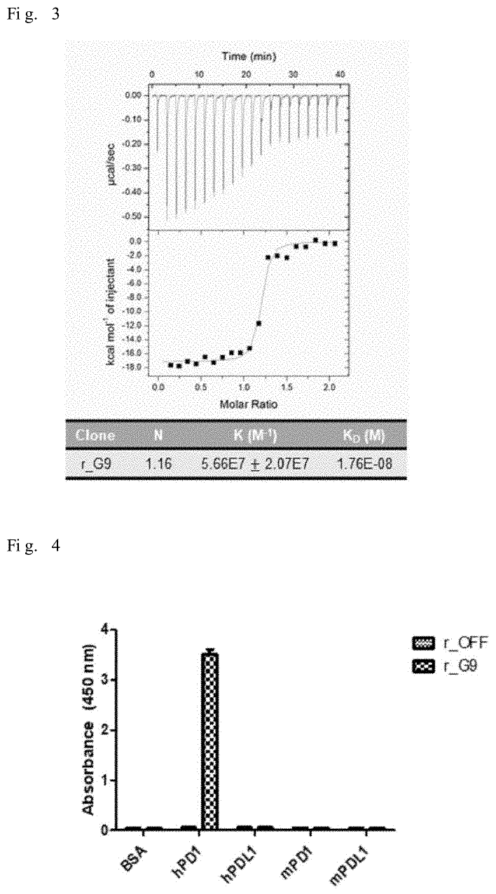

Thereafter, a mutant polypeptide having increased ability to bind to PD-1 was produced by applying mutations to the selected polypeptides in a binding affinity enhancement experiment to impart sufficient binding affinity. To this end, a second library in which amino acids at positions 137, 139, 141 and 142 are mutated in the amino acid sequence encoded by the DNA sequence of SEQ ID NO: 1 was constructed, and a phage display method using the second library was performed, whereby a novel polypeptide (SEQ ID NO: 4) having high ability to bind to PD-1 was secondarily selected (FIG. 3).

Accordingly, an aspect of the present invention relates to a polypeptide selectively binding to programmed cell death protein 1 (PD-1), in which the N-terminus of an LRR (leucine-rich repeat) family protein having an alpha-helical capping motif, the modified repeat module of a VLR (variable lymphocyte receptor) protein, and the C-terminus of the VLR protein are fused, including one or more amino acid mutations selected from the group consisting of, in the amino acid sequence represented by SEQ ID NO: 2, i) substitution of isoleucine at position 91 with asparagine, ii) substitution of threonine at position 93 with tryptophan, iii) substitution of glycine at position 94 with glutamic acid, iv) substitution of valine at position 115 with threonine, v) substitution of valine at position 117 with phenylalanine, vi) substitution of glutamic acid at position 118 with leucine, vii) substitution of alanine at position 141 with lysine, and viii) substitution of histidine at position 142 with phenylalanine.

In the present invention, the N-terminus of the LRR family protein having the alpha-helical capping motif may be the N-terminus of the internalin protein, and the internalin protein is preferably selected from the group consisting of internalin A, internalin B, internalin C, internalin H, and internalin J, and is more preferably an internalin B protein.

As used herein, the term “internalin B protein” refers to a type of LRR family protein expressed in a Listeria strain, and is known to be stably expressed even in microorganisms because the N-terminal structure is different from other LRR family proteins, in which other hydrophobic cores are uniformly distributed throughout the molecule. The N-terminus of the internalin protein may be effectively used for stable expression of LRR family proteins in microorganisms because the most important N-terminal site for folding the repeat module is derived from microorganisms, and also because the shape thereof has a more stable structure including an alpha helix.

As used herein, the term “N-terminus of internalin protein” refers to the N-terminus of an internalin protein required for water-soluble expression and folding of the protein, which is an alpha-helical capping motif and a repeat module of the internalin protein. The N-terminus of the internalin protein includes, without limitation, the N-terminus of the internalin protein required for water-soluble expression and folding of the protein, examples thereof including an alpha-helical capping motif “ETITVSTPIKQIFPDDAFAETIKANLKKKSVTDAVTQNE” (SEQ ID NO: 7) and a repeat module. Preferably, the repeat module pattern includes “L××L××L×L××N” (SEQ ID NO: 8). In the repeat module pattern, L represents alanine, glycine, phenylalanine, tyrosine, leucine, isoleucine, valine, or tryptophan, N represents asparagine, glutamine, serine, cysteine, or threonine, and x represents a hydrophilic amino acid. For the N-terminus of the internalin protein, an N-terminus having high structural similarity may be selected depending on the type of LRR family protein that is fused thereto, and the most stable amino acid is selected through calculation of binding energy, etc., so amino acid mutation in the corresponding module is possible.

As used herein, the term “variable lymphocyte receptor (VLR)” refers to a type of LRR family protein expressed in hagfish and lampreys. It is a protein that performs immune functions in hagfish and lampreys, and may be useful as a frame capable of binding to various antigenic substances. The polypeptide in which the N-terminus of the internalin B protein and the VLR protein are fused has higher water solubility and expression level than the VLR protein to which the internalin B protein is not fused, so it may be used for the production of novel protein therapeutics based thereon.

As used herein, the term “LRR (leucine-rich repeat) protein” refers to a protein formed by a combination of modules in which leucine is repeated at a certain position, (i) at least one LRR repeat module is provided, (ii) the LRR repeat module consists of 20 to 30 amino acids, and (iii) the LRR repeat module has “L××L××L×L××N” as a conserved pattern, in which L represents hydrophobic amino acids, such as alanine, glycine, phenylalanine, tyrosine, leucine, isoleucine, valine, and tryptophan, N represents asparagine, glutamine, serine, cysteine, or threonine, and x represents any of 20 amino acids, and (iv) the LRR family protein is a protein having a three-dimensional structure resembling a horseshoe. The LRR family protein of the present invention may include all mutants having not only sequences that are already known or found using newly induced mRNA or cDNA in vivo, but also sequences that are not known in nature, obtained through consensus design or the like, and having a frame of the repeat module.

As used herein, the term “repebody” refers to a polypeptide optimized by fusing the N-terminus of the internalin having the LRR structure with VLR based on the structural similarity therebetween so as to obtain a consensus design. The repebody protein may be structurally divided into a concave region and a convex region. Here, it is known that the concave region has high sequence diversity and is important for protein interaction. In contrast, the convex region plays a role in stably maintaining the overall structure of the protein based on the highly conserved sequence. The repebody protein may include all fusion LRR family proteins in which water-soluble expression and biophysicochemical properties of the protein are improved, obtained from all proteins belonging to the LRR family having the repeat module using the above method.

In the present invention, the polypeptide may be represented by the amino acid sequence of SEQ ID NO: 3 or 4.

In the present invention, the polypeptide may bind to programmed cell death protein 1 to thus inhibit the activity thereof.

Another aspect of the present invention relates to a polynucleotide encoding the polypeptide.

In the present invention, the polynucleotide may be, but is not particularly limited to, a polynucleotide encoding the amino acid sequence of any one of SEQ ID NOS: 3 and 4, and may be a polynucleotide having a nucleotide sequence having homology of 70% or more, preferably 80% or more, and more preferably 90% or more with the polynucleotide described above, but the present invention is not particularly limited thereto.

Still another aspect of the present invention relates to a recombinant vector including the polynucleotide.

As used herein, the term “vector” refers to a DNA product containing the nucleotide sequence of a polynucleotide encoding a target protein operably linked to an appropriate regulatory sequence so that the target protein is capable of being expressed in an appropriate host. The regulatory sequence includes a promoter capable of initiating transcription, any operator sequence for regulating transcription, a sequence encoding an appropriate mRNA ribosome-binding site, and a sequence regulating termination of transcription and decoding. After transformation into an appropriate host, the vector is able to replicate or function regardless of the host genome, and may be integrated into the genome itself.

The vector that is used in the present invention is not particularly limited, so long as it is able to replicate in a host, and any vector known in the art may be used. Examples of commonly used vectors include natural or recombinant plasmids, phagemids, cosmids, viruses, and bacteriophages. For example, phage vectors or cosmid vectors may include pWE15, M13, λMBL3, λMBL4, λIXII, λASHII, λAPII, λt10, λt11, Charon4A, and Charon21A, and plasmid vectors may include pBR-, pUC-, pBluescriptII-, pGEM-, pTZ-, pCL-, and pET-based plasmid vectors. The vector that is used in the present invention is not particularly limited, and a known expression vector may be used. Preferably, pACYC177, pACYC184, pCL, pECCG117, pUC19, pBR322, pMW118, pCC1BAC, pET-21a, and pET-32a vectors and the like are used. Most preferably, a pET-21a or pET-32a vector is used.

Yet another aspect of the present invention relates to a recombinant microorganism into which the polynucleotide or the recombinant vector including the polynucleotide is introduced.

As used herein, the term “recombinant microorganism” refers to a transfected cell in which a vector having a gene encoding at least one target protein is introduced into a host cell so as to express the target protein, and may include all cells, such as eukaryotic cells, prokaryotic cells, and the like, and examples thereof may include bacterial cells such as E. coli, Streptomyces, Salmonella typhimurium, etc.; yeast cells; fungal cells such as Pichia pastoris, etc.; insect cells such as Drosophila, Spodoptera Sf9 cells, etc.; animal cells such as CHO, COS, NSO, 293, bow melanoma cells, etc.; and plant cells, but the present invention is not particularly limited thereto. The host cell that is used in the present invention is not particularly limited, but preferably E. coli is used as the host cell. Most preferably, E. coli BL21 (DE3) or OrigamiB (DE3) is used as the host cell.

As used herein, the term “transformation” refers to introduction of a vector including a polynucleotide encoding a target protein into a host cell so that the protein encoded by the polynucleotide is expressed in the host cell. Any transformed polynucleotide may be used, regardless of whether it is inserted and located into the chromosome of the host cell or is located extrachromosomally, so long as it is able to be expressed in the host cell. In addition, the polynucleotide includes DNA and RNA encoding a target protein. The polynucleotide may be introduced in any form without limitation, so long as it is able to be introduced into the host cell and expressed. For example, the polynucleotide may be introduced into a host cell in the form of an expression cassette, which is a gene construct including all factors necessary for self-expression. The expression cassette typically includes a promoter, a transcription termination signal, a ribosome-binding site, and a translation termination signal, operably linked to the polynucleotide. The expression cassette may take the form of an expression vector capable of self-replication. In addition, the polynucleotide may be introduced into a host cell in its own form and operably linked to a sequence required for expression in the host cell.

Still yet another aspect of the present invention relates to a method of producing the polypeptide of the present invention including (i) expressing the polypeptide by culturing the recombinant microorganism and (ii) recovering the polypeptide from the cultured recombinant microorganism or a culture.

In the present invention, culturing the recombinant microorganism is preferably performed through, but is not particularly limited to, a known batch culture method, a continuous culture method, a fed-batch culture method, etc., and for the culture conditions, an appropriate pH (pH 5-9, preferably pH 6-8, most preferably pH 6.8) may be set using a basic compound (e.g. sodium hydroxide, potassium hydroxide, or ammonia) or an acidic compound (e.g. phosphoric acid or sulfuric acid), aerobic conditions may be maintained by introducing oxygen or an oxygen-containing gas mixture into the culture, and culture is preferably carried out at a culture temperature of 20 to 45° C., preferably 25 to 40° C., for about 10 to 160 hours, but the present invention is not particularly limited thereto. The polypeptide produced through culture may be secreted into the medium or may remain in the cell.

For the culture medium that is used, the carbon source may include sugars and carbohydrates (e.g. glucose, sucrose, lactose, fructose, maltose, molasses, starch, and cellulose), oils and fats (e.g. soybean oil, sunflower seed oil, peanut oil, and coconut oil), fatty acids (e.g. palmitic acid, stearic acid, and linoleic acid), alcohols (e.g. glycerol and ethanol), and organic acids (e.g. acetic acid), which may be used alone or in combination, the nitrogen source may include nitrogen-containing organic compounds (e.g. peptone, yeast extract, broth, malt extract, corn steep liquor, soybean meal powder, and urea) or inorganic compounds (e.g. ammonium sulfate, ammonium chloride, ammonium phosphate, ammonium carbonate, and ammonium nitrate), which may be used alone or in combination, the phosphorus source may include potassium dihydrogen phosphate, dipotassium hydrogen phosphate, sodium-containing salts corresponding thereto, etc., which may be used alone or in combination, and essential growth-promoting materials such as other metal salts (e.g. magnesium sulfate or iron sulfate), amino acids, and vitamins may be included.

To recover the polypeptide produced through culture in the present invention, a desired polypeptide may be collected from the culture solution using an appropriate method known in the art depending on the culture method, for example, a batch culture method, a continuous culture method, or a fed-batch culture method.

A further aspect of the present invention relates to a composition for preventing or treating cancer containing the polypeptide as an active ingredient.

In the present invention, prevention or treatment of cancer may be achieved through the polypeptide binds to programmed cell death protein 1 of T cells.

In the present invention, prevention or treatment of cancer may be achieved through the polypeptide binds to programmed cell death protein 1.

As used herein, the term “cancer” or “tumor” refers to a mass that has grown abnormally due to the autonomous overgrowth of body tissue. The cancer or tumor in the present invention may be of any type that expresses PD-L1, and is preferably selected from the group consisting of non-Hodgkin's lymphoma, Hodgkin's lymphoma, acute myeloid leukemia, acute lymphoid leukemia, multiple myeloma, head and neck cancer, lung cancer, non-small cell lung cancer, glioblastoma, colorectal/rectal cancer, pancreatic cancer, breast cancer, ovarian cancer, malignant melanoma, prostate cancer, kidney cancer, and mesothelioma, but is not limited thereto.

Still a further aspect of the present invention relates to a method of treating or preventing cancer including administering the polypeptide or a pharmaceutically acceptable salt thereof.

Yet a further aspect of the present invention relates to the use of the polypeptide or a pharmaceutically acceptable salt thereof for the treatment or prevention of cancer.

Still yet a further aspect of the present invention relates to the use of the polypeptide or a pharmaceutically acceptable salt thereof for the manufacture of a medicament for the treatment or prevention of cancer.

In the present invention, prevention or treatment of cancer may be characterized in that the polypeptide binds to programmed cell death protein 1 of T cells.

In the present invention, prevention or treatment of cancer may be characterized in that the polypeptide binds to programmed cell death protein 1.

As used herein, the term “cancer” or “tumor” refers to a mass that has grown abnormally due to the autonomous overgrowth of body tissue. The cancer or tumor in the present invention may be of any type that expresses PD-L1, and is preferably selected from the group consisting of non-Hodgkin's lymphoma, Hodgkin's lymphoma, acute myeloid leukemia, acute lymphoid leukemia, multiple myeloma, head and neck cancer, lung cancer, non-small cell lung cancer, glioblastoma, colorectal/rectal cancer, pancreatic cancer, breast cancer, ovarian cancer, malignant melanoma, prostate cancer, kidney cancer, and mesothelioma, but is not limited thereto.

As used herein, the term “treatment” means treating cancer or preventing the progression of cancer by reversing the symptoms of a disease as well as suppressing or alleviating cancer or one or more symptoms caused therefrom by administration of the composition. As used herein, the term “prevention” refers to any action that inhibits cancer or delays the onset of cancer by administration of the composition. In the present invention, it is possible to prevent or treat cancer because the polypeptide obtained in the present invention binds to programmed cell death protein 1, and the polypeptide binds to programmed cell death protein 1 to thus significantly inhibit the activity thereof, so cancer may be prevented or treated.

The composition for preventing or treating cancer containing the polypeptide of the present invention as an active ingredient may further include a pharmaceutically acceptable carrier, and may be formulated together with the carrier.

As used herein, the term “pharmaceutically acceptable carrier” refers to a carrier or diluent that does not inhibit the biological activity or properties of the administered compound and does not stimulate an organism. Examples of the pharmaceutically acceptable carrier in a composition formulated as a liquid solution include those that are sterile and biocompatible, such as saline, sterile water, Ringer's solution, buffered saline, albumin injection solution, dextrose solution, maltodextrin solution, glycerol, ethanol, and mixtures of one or more of these components, and other typical additives such as antioxidants, buffers, bacteriostats and the like may be added as needed. Moreover, the composition of the present invention may be formulated in the form of pills, capsules, granules or tablets, as well as injectable forms such as an aqueous solution, suspension, emulsion, etc., by adding diluents, dispersants, surfactants, binders, and lubricants.

The composition for preventing or treating cancer containing the polypeptide of the present invention and the pharmaceutically acceptable carrier is applicable to any formulation containing the same as an active ingredient, and may be prepared as an oral or parenteral formulation. The pharmaceutical formulation of the present invention may take any form suitable for oral, rectal, nasal, topical (including buccal and sublingual), subcutaneous, vaginal, or parenteral (including intramuscular, subcutaneous, and intravenous) administration, or any form suitable for administration by inhalation or insufflation.

Examples of formulations for oral administration containing the composition of the present invention as an active ingredient may include tablets, troches, lozenges, aqueous or oily suspensions, prepared powders or granules, emulsions, hard or soft capsules, syrups, and elixirs. For formulation into dosage forms such as tablets and capsules, a binder such as lactose, saccharose, sorbitol, mannitol, starch, amylopectin, cellulose, or gelatin, an excipient such as dicalcium phosphate, a disintegrant such as corn starch or sweet potato starch, or a lubricant such as magnesium stearate, calcium stearate, sodium stearyl fumarate, or polyethylene glycol wax may be included, and for capsule formulations, a liquid carrier such as fatty oil may be further contained, in addition to the above-mentioned materials.

Formulations for parenteral administration containing the composition of the present invention as an active ingredient may include injectable forms for subcutaneous injection, intravenous injection or intramuscular injection, suppositories for a suppository injection method, or sprays such as aerosols for inhalation through the respiratory organs. In order to prepare an injectable formulation, the composition of the present invention may be mixed with a stabilizer or buffer in water to afford a solution or suspension, which may be formulated in unit dosage forms such as ampoules or vials. A suppository formulation may include a composition for rectal administration such as a suppository or an enema containing a typical suppository base such as cocoa butter or other glycerides. For a spray formulation, such as an aerosol, the additives may be blended with a propellant or the like to disperse the water-dispersed concentrate or wet powder.

Even yet a further aspect of the present invention relates to a method of preventing or treating cancer including administering the composition for preventing or treating cancer containing the polypeptide as an active ingredient.

As used herein, the term “administration” means introducing the pharmaceutical composition of the present invention to a patient through any appropriate method. The composition of the present invention may be administered through various routes, either oral or parenteral, so long as it is able to reach target tissue, and specifically may be administered in a typical manner via oral, rectal, topical, intravenous, intraperitoneal, intramuscular, intraarterial, transdermal, intranasal, inhalation, intraocular, or intradermal routes.

The treatment method of the present invention includes administering the composition for preventing or treating cancer of the present invention in a pharmaceutically effective amount. It will be apparent to those skilled in the art that an appropriate total daily dosage may be determined by a doctor within the scope of medical judgment. A specific therapeutically effective amount for a certain patient is preferably dependent on various factors including the type and extent of the response to be achieved, whether the specific composition includes other agents, the patient's age, body weight, general health status, gender, and diet, the time of administration, the route of administration, the secretion rate of the composition, the duration of treatment, and drugs used together or concurrently with the specific composition, and similar factors well-known in the pharmaceutical field. Therefore, it is preferable to determine the effective amount of the composition for preventing or treating cancer suitable for the purpose of the present invention in consideration of the foregoing.

In addition, the treatment method of the present invention is applicable to any animal in which disease such as tumor development and angiogenesis may occur due to excessive activity of programmed cell death protein 1, and the animal includes not only humans and primates, but also livestock such as cattle, pigs, sheep, horses, dogs, and cats.

A better understanding of the present invention may be obtained through the following examples. These examples are merely set forth to illustrate the present invention, and are not to be construed as limiting the scope of the present invention, as will be apparent to those skilled in the art.

### Example 1: Construction of Repebody Library Based on Protein Structure

A repebody has modularity in which repeat units having a conserved leucine sequence are continuously connected to maintain the overall protein structure, and a structural characteristic classified into a concave region and a convex region due to curvature of the shape of the overall structure, like LRR proteins present in nature. A hypervariable region, like the complementarity-determining region (CDR) of an antibody, is located in the concave region to thus mediate protein-protein interaction. In addition, the convex region plays an important role in maintaining the entire structure of LRR based on the well-conserved sequence thereof. The protein structure of the repebody was analyzed, and a random library was designed in the following manner.

Specifically, a random mutation library was constructed by selecting six amino acid residues at positions 91, 93, 94, 115, 117, and 118 located in the concave region of two consecutive modules (LRRV2, LRRV3 module) located in the direction of the amine group terminal (FIG. 1).

Then, mutagenic primers for library construction were synthesized so that the selected amino acid was substituted with an NNK degenerate codon. The primer sequences were as follows.

M1:
SEQ ID NO: 5
CTG CCG AAT GGC GTC TTT GAT AAA CTG ACG AAC CTG
AAA GAA CTG NNK CTG NNK NNK AAT CAA CTG CAG TCT
CTG CCG GAC
M2:
SEQ ID NO: 6
CGT CAG TTT ATC AAA GAC GCC ATT CGG CAG GCT CTG
CAG TTG GTT MNN MNN CAG MNN CAG ATA CGT CAG ATT
GGT CAG TTC

Then, using the primers, an overlap polymerase chain reaction (overlap PCR) for both modules was performed to afford library DNA, which was then inserted into the phagemid pBEL118M of SEQ ID NO: 2, described in a patent previously granted to the present inventors (Korea Patent No. 1,356,075), to obtain a final library phagemid.

The library thus obtained was introduced into E. coli TG-1 through electroporation to obtain a transformant, whereby a library having diversity at a level of 2.0×10⁸ was constructed.

### Example 2: Selection of Polypeptide Specifically Binding to PD-1 Through Random Phage Library

### Example 2-1: Selection of Polypeptide Binding to PD-1 Through Panning of Repebody Library

Using the library constructed in Example 1, a polypeptide capable of binding to PD-1 was selected and purified. In order to select candidates capable of binding to PD-1, biotinylated PD-1 was added at a concentration of 10 μg/ml to streptavidin-conjugated magnetic beads, followed by a binding reaction at room temperature for 30 minutes and then washing three times with PBS. The PD-1-bound magnetic beads and magnetic beads for negative selection were blocked with a PBS solution (TPBSA) containing 1% BSA and 0.05% Tween 20 at room temperature for 1 hour. Then, the purified phage was added at a concentration of 10¹² cfu/ml to the magnetic beads for negative selection and allowed to react at room temperature for 1 hour, after which the phage randomly binding to the magnetic beads was removed. Thereafter, only the phage present in the supernatant was added to the PD-1-bound magnetic beads and allowed to react at room temperature for 1 hour. After completion of the reaction, the reaction product was washed five times with a PBS solution (TPBS) containing 0.05% Tween 20 for a total of 1 minute, and was then washed once with PBS. Finally, 1 ml of 0.2 M glycine-HCl (pH 2.2) was added to the immunotube and allowed to react at room temperature for 10 minutes, so a phage on the surface of which a candidate repebody capable of binding to PD-1 was expressed was eluted. The eluate was neutralized with 100 μl of 1.0 M Tris-HCl (pH 9.0), added to 8.9 ml of a TG-1 solution of E. coli (OD600=0.5) as a host cell, and then spread on a 2×YT plate. This biopanning process was repeated five times in the same manner. As a result, it was confirmed that the phage specifically binding to PD-1 was enriched through each panning process. The above results were interpreted to mean that the amount of the library phage that was specifically bound to PD-1 was increased.

### Example 2-2: Confirmation of Specific Binding of Selected Repebody to PD-1 and Sequence Analysis

The phages selected through the method of Example 2-1 were subjected to enzyme-linked immunosorbent assay (ELISA) using a 96-well plate coated with BSA and PD-1. 10 repebody candidates having an absorbance (OD450) of PD-1, at least 4.9 times as high as that of BSA, were selected, and individual amino acid sequences thereof were analyzed, based on which it was confirmed that all of the repebody clones obtained from the selected phage had the same sequence (SEQ ID NO: 3). It was confirmed that, in the selected A1 repebody, isoleucine, which is the amino acid at position 91, was substituted with asparagine, threonine at position 93 was substituted with tryptophan, glycine at position 94 was substituted with glutamic acid, valine at position 115 was substituted with threonine, valine at position 117 was substituted with phenylalanine, and glutamic acid at position 118 was substituted with leucine. The dissociation constant of the selected A1 repebody to PD-1 was measured through isothermal titration calorimetry (ITC), and indicated binding affinity of 617 nM (FIG. 2).

### Example 3: Increase in Binding Affinity of Repebody to PD-1 Using Module-Based Method

The module-based affinity enhancement method described in the present invention was performed according to a method disclosed in a prior patent (KR2012-0019927). This method is a technique that is usually carried out for a protein having a repeat module, and has been successfully reproduced in the present patent, enabling the design of proteins having high affinity.

### Example 3-1: Additional Library Construction Using Module and Confirmation of Increased Binding Affinity and Cross-Linking to Other Proteins

An attempt was made to develop a mutant having increased binding affinity through an additional library construction method using the module used in the prior patent (KR2012-0019927).

Specifically, a library for module-based affinity enhancement was obtained by mutating a total of four amino acid residues of an LRRV4 module, that is, amino acids at positions 137, 139, 141, and 142. This library was subjected to panning a total of 3 times to obtain a repebody G9 clone having increased binding affinity (SEQ ID NO: 4).

In the G9 clone, in the amino acid encoded by the DNA of SEQ ID NO: 1, isoleucine, which is the amino acid at position 91, was substituted with asparagine, threonine at position 93 was substituted with tryptophan, glycine at position 94 was substituted with glutamic acid, valine at position 115 was substituted with threonine, valine at position 117 was substituted with phenylalanine, glutamic acid at position 118 was substituted with leucine, alanine at position 141 was substituted with lysine, and histidine at position 142 was substituted with phenylalanine.

The dissociation constant of the selected G9 repebody was measured through isothermal titration calorimetry, and indicated high binding affinity of 17 nM (FIG. 3).

In order to confirm whether the selected G9 repebody specifically exhibits strong binding only to human PD-1, an experiment was performed to determine whether cross-linking occurs with other proteins having a sequence and structure similar to that of human PD-1. Other proteins for evaluating cross-linking were mouse PD-1 protein, which is structurally identical to human PD-1 and has at least 60% amino acid sequence homology, and human and mouse PD-1 receptors (PD-L1).

Based on the results of measuring the extent of binding through an enzyme-linked immunosorbent assay (ELISA) using the G9 repebody after coating a 96-well plate with human and mouse PD-1 and PD-L1 proteins, it was confirmed that the selected G9 repebody specifically bound only to human PD-1 (FIG. 4).

Through these results, the present inventors ascertained that a repebody having high binding affinity to human PD-1 was successfully obtained, and identified the same to be a polypeptide having specific binding affinity to PD-1.

### Example 4: Identification of Antigenic Determinant of Selected Repebody

It is very important to select a repebody that binds to a site where PD-1 interacts with a PD-1 receptor, namely PD-L1, among the repebodies that bind to PD-1. Currently, an anti-PD-1 monoclonal antibody binds to the above site to thus prevent PD-1 from interacting with the PD-1 receptor, thereby effectively inhibiting the activity of PD-1, ultimately exhibiting high therapeutic efficacy.

Against this background, competitive enzyme-linked immunosorbent assay (competitive ELISA) was performed to determine whether the finally obtained G9 repebody is able to inhibit the interaction between PD-1 and PD-L1. Specifically, whether the amount of PD-L1 that bound to PD-1 decreased due to competitive binding of the repebody was determined by adding the repebody and soluble PD-L1 to the PD-1-coated plate at the same time.

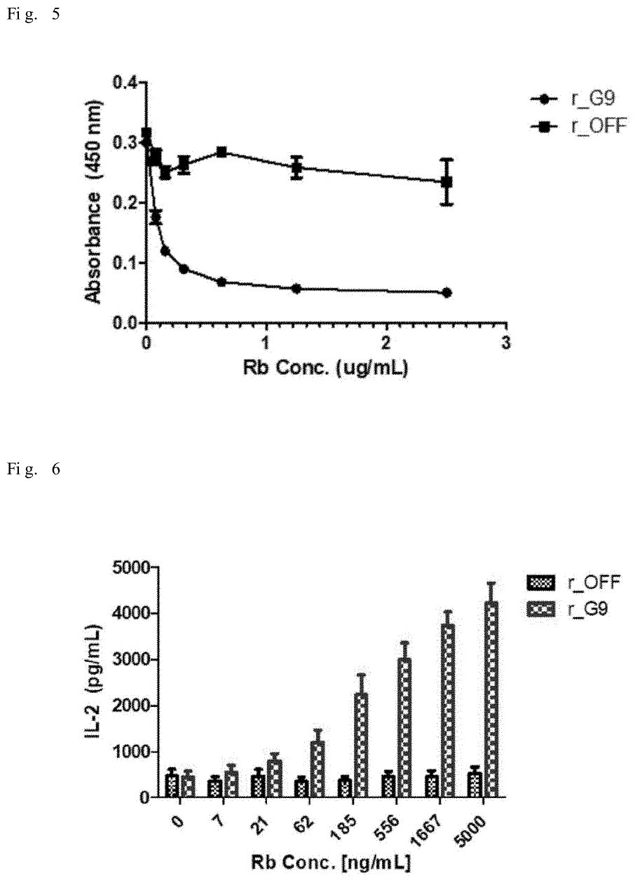

As a result, it was confirmed that the signal of PD-L1 decreased only upon treatment with the selected G9 repebody, indicating that the antigenic determinant of the G9 repebody is the site where PD-1 interacts with the PD-1 receptor (FIG. 5).

### Example 5: Analysis of PD-1 Signaling Inhibition Potential of Selected Repebody

PD-1 bound to PD-L1 is known to block the signaling pathway that activates T cells by attracting a phosphatase called SHP2 while being phosphorylated. The selected G9 repebody must not only specifically bind to PD-1, but must also prevent PD-1 signaling by blocking interaction with PD-L1, ultimately activating T cells.

To this end, T cells obtained from human blood were mixed with allogeneic dendritic cells to induce an immune response, and then whether T cells activated by the G9 repebody were able to produce and secrete interleukin-2 (IL-2) and interferon-gamma (IFN-γ) as cytokines was tested.

Specifically, blood from healthy persons was provided for experimentation (the subjects were recruited with the permission of the KAIST Institutional Animal Care and Use Committee and then blood was directly collected at the KAIST Clinic), and peripheral blood mononuclear cells (PBMC) were isolated from the blood. Peripheral blood mononuclear cells were isolated using Ficoll and centrifugation. T cells were isolated and separately obtained using a kit (a Dynabeads CD4 positive isolation kit, Invitrogen, USA) for specifically isolating T cells from peripheral blood mononuclear cells.

Meanwhile, monocytes were isolated using a kit (Monocyte isolation kit II, Miltenyi Biotec, Germany) for isolating peripheral blood mononuclear cells from blood obtained from other blood donors in the same manner as above and specifically isolating monocytes therefrom. The isolated monocytes were treated with cytokines such as granulocyte-macrophage colony-stimulating factor (GM-CSF), interleukin-4, and tumor necrosis factor-alpha (TNF-α) to thus induce differentiation into dendritic cells.

A mixed lymphocyte reaction (MLR) experiment was performed by mixing and culturing the obtained 1×10⁵ T cells and 1×10⁴ allogeneic dendritic cells in a 96-well cell culture plate. Each well was treated with the repebody at different concentrations followed by culture for five days, after which the amounts of cytokines present in the supernatant were measured through an enzyme-linked immunosorbent assay.

Based on the results thereof, it was confirmed that, when the G9 repebody was used together, the amounts of interleukin-2 and interferon-gamma, which are indicators of T-cell activation, increased in proportion to the concentration of the G9 repebody (FIGS. 6 and 7).

### Example 6: Confirmation of Cancer Cell Growth Inhibitory Activity of Selected Repebody in Mouse Model

In order to evaluate whether the selected repebody binds to PD-1 of T cells in the body and activates T cells to actually kill cancer cells, an in-vivo experiment was performed using an immune-deficient mouse model. 2×10⁶ NCI-H292 cancer cell lines (Korea Cell Line Bank) and 5×10⁵ human peripheral blood mononuclear cells were mixed with BD Matrigel Matrix to prepare a total volume of 100 μl, and the resulting mixture was injected subcutaneously into the right flank of female NOD/ShiLtJ-Rag2em1AMC Il2rgem1AMC (NRGA) mice 6-8 weeks old (Junga Bio). The repebody was intraperitoneally injected at a dose of 10 mg/kg starting one day after inoculation. The repebody was administered a total of four times, on the 1^{st}, 3^{rd}, 7^{th}, and 10^{th} days after inoculation. As control groups, a group injected with off-target repebody and a group injected only with DPBS were used. The tumor size of mice was measured using a caliper once every 3-4 days starting one week after inoculation. The tumor volume was calculated using the following formula.

Tumor volume (V)=(tumor area (W)²×tumor length (L))/2(mm³).

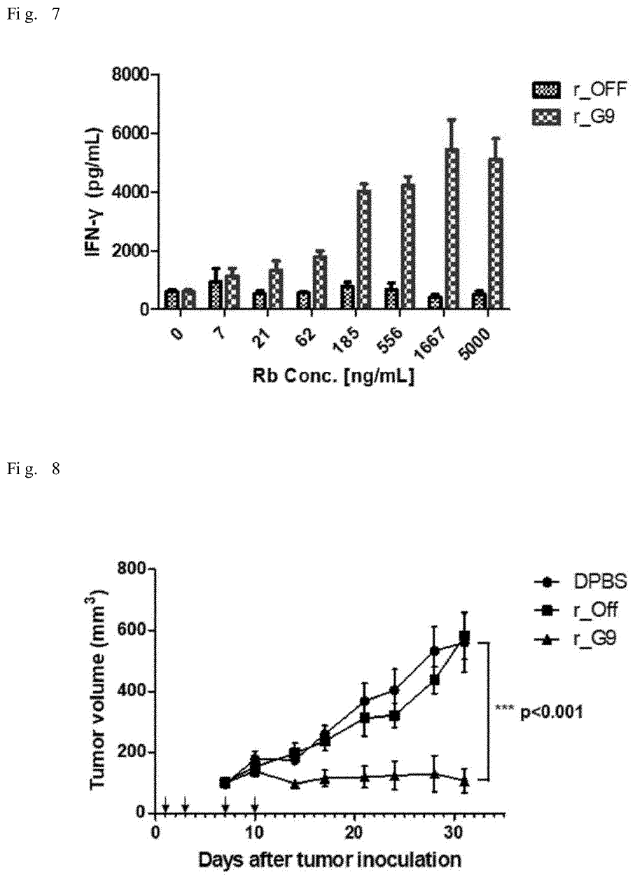

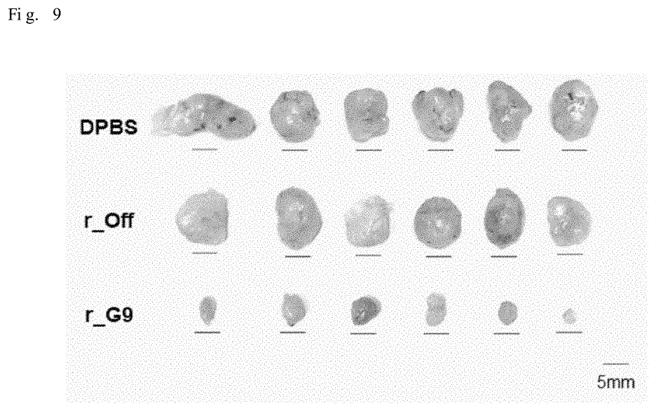

Based on the results thereof, it was confirmed that tumor growth was inhibited in the mouse group administered with the G9 repebody. In addition, it was confirmed that this effect was maintained until day 31 (FIGS. 8 and 9)

### INDUSTRIAL APPLICABILITY

The novel polypeptide binding to PD-1 according to the present invention not only specifically binds to PD-1, but also inhibits the activity thereof, thus inducing immune T-cell activity, and is useful for the development of immunotherapeutic agents for various diseases associated with PD-1.

Although specific embodiments of the present invention have been disclosed in detail above, it will be obvious to those of ordinary skill in the art that the description is merely of preferable exemplary embodiments and is not to be construed as limiting the scope of the present invention. Therefore, the substantial scope of the present invention is to be defined by the appended claims and equivalents thereof.

### Sequence List Free Text

An electronic file is attached.

1. A polypeptide selectively binding to programmed cell death protein 1 (PD-1), in which an N-terminus of an LRR (leucine-rich repeat) family protein having an alpha-helical capping motif, a modified repeat module of a VLR (variable lymphocyte receptor) protein, and a C-terminus of the VLR protein are fused, comprising one or more amino acid mutations selected from the group consisting of, in an amino acid sequence represented by SEQ ID NO: 2:
i) substitution of isoleucine at position 91 with asparagine;
ii) substitution of threonine at position 93 with tryptophan;
iii) substitution of glycine at position 94 with glutamic acid;
iv) substitution of valine at position 115 with threonine;
v) substitution of valine at position 117 with phenylalanine;
vi) substitution of glutamic acid at position 118 with leucine;
vii) substitution of alanine at position 141 with lysine; and
viii) substitution of histidine at position 142 with phenylalanine.

2. The polypeptide according to claim 1, wherein the N-terminus of the LRR family protein having the alpha-helical capping motif is an N-terminus of an internalin protein.

3. The polypeptide according to claim 2, wherein the internalin protein is selected from the group consisting of internalin A, internalin B, internalin C, internalin H, and internalin J.

4. The polypeptide according to claim 3, wherein the internalin protein is an internalin B protein.

5. The polypeptide according to claim 1, wherein the modified repeat module of the VLR protein comprises a repeat module pattern below:
L××L××L×L××N
in the pattern,
L is alanine, glycine, phenylalanine, tyrosine, leucine, isoleucine, valine, or tryptophan;
N is asparagine, glutamine, serine, cysteine, or threonine, and
x is any of 20 amino acids.

6. The polypeptide according to claim 1, wherein the polypeptide is represented by an amino acid sequence of SEQ ID NO: 3 or 4.

7. The polypeptide according to claim 1, wherein the polypeptide binds to programmed cell death protein 1 and inhibits activity thereof.

8. A polynucleotide encoding the polypeptide according to claim 1.

9. A recombinant vector comprising the polynucleotide according to claim 8.

10. A recombinant microorganism into which the recombinant vector according to claim 9 is introduced.

11. A method of producing a polypeptide specifically binding to programmed cell death protein 1 according to claim 1, comprising:
(i) expressing the polypeptide by culturing a recombinant microorganism into which a recombinant vector comprising a polynucleotide encoding the polypeptide is introduced; and
(ii) recovering the polypeptide from the cultured recombinant microorganism or a culture.

12. A method of preventing or treating cancer including administering the polypeptide according to claim 1 to a patient.

13. The method according to claim 12, wherein the cancer is selected from the group consisting of non-Hodgkin's lymphoma, Hodgkin's lymphoma, acute myeloid leukemia, acute lymphoid leukemia, multiple myeloma, head and neck cancer, lung cancer, non-small cell lung cancer, glioblastoma, colorectal/rectal cancer, pancreatic cancer, breast cancer, ovarian cancer, malignant melanoma, prostate cancer, kidney cancer, and mesothelioma.
